(19) United States
(12) Patent Application Publication
(10) Pub. No.: US 2022/0218761 A1
(43) Pub. Date: Jul. 14, 2022

(54) MONITORING AND ALTERING THE GUT MICROBIOME IN DISEASE

(71) Applicant: The General Hospital Corporation, Boston, MA (US)

(72) Inventors: Maureen M. Leonard, Boston, MA (US); Alessio Fasano, Boston, MA (US); Victoria Kenyon, Cambridge, MA (US); Ali Zomorrodi, Auburndale, MA (US)

(21) Appl. No.: 17/609,137

(22) PCT Filed: May 6, 2020
(86) PCT No.: PCT/US20/31694

§ 371 Date: Nov. 5, 2021

Related U.S. Application Data
(60) Provisional application No. 62/844,045, filed on May 6, 2019.

(51) Int. Cl. A61K 35/74; A61K 31/19; A61K 31/191; A61K 31/7008; A61K 35/742; A61K 35/745; G01N 33/68; C12Q 1/689

(52) U.S. Cl. CPC A61K 35/74; A61K 31/19; A61K 31/191; A61K 31/7008; A61K 2035/115; A61K 35/745; G01N 33/6812; C12Q 1/689; A61K 35/742

(57) ABSTRACT

Methods for predicting whether a subject will develop a chronic inflammatory condition such as celiac disease, and methods and composition comprising anti-inflammatory microbes or metabolites that can be used to treat or reduce the risk of developing such conditions.

### CLAIM OF PRIORITY

This application claims the benefit of U.S. Provisional Application Ser. No. 62/844,045, filed on 6 May 2019. The entire contents of the foregoing are incorporated herein by reference.

### FEDERALLY SPONSORED RESEARCH OR DEVELOPMENT

This invention was made with Government support under Grant Nos. DK104344 and DK122127 awarded by the National Institutes of Health. The Government has certain rights in the invention.

### TECHNICAL FIELD

Provided herein are methods for predicting whether a subject will develop a chronic inflammatory condition such as celiac disease, and methods and composition comprising anti-inflammatory microbes or metabolites that can be used to treat or reduce the risk of developing such conditions.

### BACKGROUND

It is now apparent that genetic predisposition and exposure to environmental triggers are necessary but not sufficient to develop a variety of chronic inflammatory conditions including autoimmune conditions such as celiac disease. For example, celiac disease (CD) is a chronic systemic autoimmune disorder that occurs in genetically predisposed individuals and is characterized by the loss of tolerance to dietary gluten protein. CD affects 1% of the global population and its incidence is expected to continue to increase in line with other autoimmune conditions yet its associated genes, human leukocyte antigen (HLA), and the trigger, gluten, have not changed [1]. Nevertheless, more than 30% of the population carry the predisposing gene and are exposed to gluten yet only 2-3% of them develop CD in their lifetime [2]. This suggests that other factors also contribute to CD pathogenesis.

### SUMMARY

The present invention is based on a rigorous multi-omics analysis of the gut microbiota in CD using a prospective longitudinal birth cohort study design. This analysis uncovered several microbes and metabolites whose abundance before CD onset are significantly changed compared to onset. These identified alterations point to a “march” from the preclinical stage to break of tolerance to gluten and subsequent onset of CD through complex patterns of increase in abundances of pro-inflammatory species and decrease in those of protective anti-inflammatory ones at different time points preceding the onset of the disease. These microbiome shifts may represent potential biomarkers of CD development, which can be scrutinized further to pinpoint tractable points of intervention in the gut microbiota/metabolome to restore tolerance to gluten and to prevent autoimmunity. Gut microbiota dysbiosis can be used as a predictive biomarkers of disease onset and provide novel therapeutic or preventive targets to intercept CD before its onset.

Thus provided herein are compositions comprising one, two, three, four, or more microbes listed in Table B, in a physiologically acceptable carrier and proper culture medium. In some embodiments, the composition comprises one, two, three, or more of: Bacteroides ovatus CLO3T12C18; Clostridium sp JCC; a Faecalibacterium prausnitzii, preferably Faecalibacterium prausnitzii L2_6; at least one Bifidobacterium longum strain, preferably selected from the group consisting of: Bifidobacterium longum DJO10A, Bifidobacterium longum NCC2705, Bifidobacterium longum subsp infantis CCUG 52486, Bifidobacterium longum subsp longum 1 6B, Bifidobacterium longum subsp longum 17 1B, Bifidobacterium longum subsp longum 35B, Bifidobacterium longum subsp longum 72B, Bifidobacterium longum subsp longum ATCC 55813, Bifidobacterium longum subsp longum F8, Bifidobacterium longum subsp longum GT15, and Bifidobacterium longum subsp longum KACC 91563; and at least one Bifidobacterium breve strain, preferably selected from the group consisting of Bifidobacterium breve 31L; Bifidobacterium breve 689b; Bifidobacterium breve ACS_071_V_Sch8b; Bifidobacterium breve CECT_7263; Bifidobacterium breve DPC_6330; Bifidobacterium breve HPH0326; Bifidobacterium breve JCM_7017; Bifidobacterium breve JCM 7019; and Bifidobacterium breve S27.

Also provided herein are compositions comprising one, two, three, four, or more anti-inflammatory metabolites selected from the group consisting of 2-Hydroxy-3-methylbutyric acid; Acetyl galactosamine; 2-Hydroxyisocaproic acid; Arabinonic acid; Lauric acid; 3-Hydroxyphenylacetic acid; Ribitol; Gluconic acid; Proline; Glycine; Glycerol; and Serine, in a physiologically acceptable carrier. In some embodiments, the composition comprises 2-Hydroxy-3-methylbutyric acid; Acetyl galactosamine; 2-Hydroxyisocaproic acid; and Arabinonic acid.

In some embodiments, the composition is formulated for oral administration. In some embodiments, the composition is a liquid, capsule, gel, or tablet.

The compositions provided herein can be used in a method of treating, or reducing the risk of developing, a chronic inflammatory condition.

Further provided herein are methods for treating or reducing the risk of developing a chronic inflammatory condition, the method comprising administering to a subject in need thereof an effective amount of a composition as described herein.

In some embodiments, the chronic inflammatory condition is celiac disease.

Also provided herein are methods for determining risk of developing a chronic inflammatory condition in a subject. The methods include providing a sample comprising stool from the subject; performing an assay to detect presence or level of a pro-inflammatory biomarker comprising (i) at least one, two, three, four, or more pro-inflammatory microbes listed in Table A, and/or (ii) at least one, two, or all three pro-inflammatory metabolites comprising serine, threonine, and glycolic acid; comparing the presence or level of the pro-inflammatory biomarker in the sample to the presence or level of the pro-inflammatory biomarker in a reference sample; and identifying a subject who has a presence or level of the pro-inflammatory biomarker above the reference sample as being at risk of developing the chronic inflammatory condition, e.g., within 6, 12, 18, or 24 months. In some embodiments, the pro-inflammatory microbes comprise one, two, three, four, or more of Porphyromonas sp., preferably Porphyromonas_sp_31_2; an Alistipes finegoldii species, preferably Alistipes finegoldii_DSM_17242; Alistipes_sp_HGB5; a Ruminococcus bicirculans species; Erysipelotrichaceae_bacterium_21_3; a Dialister invisus species, preferably Dialister_invisus_DSM_15470; a Veillonella parvula, preferably Veillonella_parvula_ACS_068_V_Sch12 or Veillonella_parvula_HSIVP1; a Parabacteroides sp., preferably Parabacteroides_sp_20_3, or Parabacteroides_sp_D13; a Lachnospiraceae bacterium species, preferably Lachnospiraceae_bacterium_3_1_46FAA; and/or Bifidobacterium adolescentis species, preferably Bifidobacterium_adolescentis_L2_32.

The methods can also or alternatively include performing an assay to detect presence or level of an anti-inflammatory biomarker comprising (i) at least one, two, three, four, or more anti-inflammatory microbes listed in Table B, and/or (ii) at least one, two, or more anti-inflammatory metabolites listed in Table D; comparing the presence or level of the anti-inflammatory biomarker in the sample to the presence or level of the anti-inflammatory biomarker in a reference sample; and identifying a subject who has a presence or level of the anti-inflammatory biomarker below the reference sample as being at risk of developing the chronic inflammatory condition, e.g., within 6, 12, 18, or 24 months. In some embodiments, the anti-inflammatory microbes include one, two, three, four, or more of those listed above.

In some embodiments, the method further includes treating the subject identified as being at risk to reduce the risk of developing the disease. In some embodiments, treating the subject comprises administering an effective amount of a composition as described herein. In some embodiments, the composition is administered orally. In some embodiments, the composition is a liquid, capsule, gel, or tablet.

In some embodiments, performing an assay to detect presence or level of a pro-inflammatory microbe comprises performing an assay to detect a protein or nucleic acid that identifies the microbe.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGs. 1A-B. Cross-sectional (inter-subject) analysis of microbiota features at CD onset. Cross-sectional analysis comparing cases and controls at CD onset was performed by using Mann-Whitney U (Wilcoxon Rank Sum) test and significant results are reported for (A) microbial strains (p-value<0.05) and (B) metabolites (p-value<0.05). No significant microbial species (p-value <0.05) were detected.

FIGS. 2A-B. Longitudinal (intra-subject) analysis for microbial species. A paired Wilcoxon (Wilcoxon Signed Rank) test was used to identify microbial species whose abundance differentially changes between a pre-onset time point (−18, −15, −12, −9, −6 and −3 months) and CD onset. Any species for which a statistically significant (p-value<0.05) change is observed in at least one time point in (A) cases and (B) controls is reported here. Here, we report only species for which significant changes are uniquely observed in cases or in controls.

FIGS. 3A-B. Longitudinal (intra-subject) analysis for microbial strains. Microbial strains with a statistically significant (Wilcoxon Signed Rank test, p-value<0.05) change in abundance in at least one pre-onset time point compared to CD onset in (A) cases and (B) controls. Only strains with a uniquely observed change in cases or controls are reported.

FIGS. 4A-B. Longitudinal (intra-subject) analysis for metabolites. Metabolites with a statistically significant (Wilcoxon Signed Rank test, p-value<0.05) change in abundance in at least one pre-onset time point compared to CD onset in (A) cases and (B) controls. Similarly, only metabolites with a uniquely observed change in cases or controls are reported.

### DETAILED DESCRIPTION

Inflammatory processes underlying CD involve both the innate and adaptive immune systems [3]. While the adaptive immune response in CD has been well described, less is known about the innate immune response following gluten exposure, which drives the early steps in CD pathogenesis and eventually leads to the loss of gluten tolerance [4]. Previous work has linked the trigger of CD, gluten, the intestinal microbiota, and the innate immune response [5-8].

Given the cross-talk between the gut microbiota and the immune system, alterations in the gut microbiota have been linked to several autoimmune conditions [9] such as inflammatory bowel disease [10], type 1 diabetes (T1D) [11], multiple sclerosis [12] and CD [13-18]. Several studies have looked for changes in the gut microbiota of infants at-risk of CD [15, 17, 18]. For example, 16S rRNA amplicon sequencing was used to detect a higher abundance of Lactobacillus until 12 months of age in one infant who later developed CD as compared to 15 at-risk infants who did not [15]. Other studies of the gut microbiota and CD have assessed changes in the first year after birth in the gut microbiota composition of individuals who later developed CD compared to controls [17, 18]. For example, Olivares et al [17] used a prospective cohort of 200 infants at risk of CD to compare, using 16S rRNA sequencing data, the intestinal microbiota of 10 cases that developed CD during the five year study period and 10 matched controls at 4 and 6 months of age. They report increases in the abundances of Firmicutes, Enterococcaceae, and Peptostreptococcaceae in controls from 4 to 6 months [17]. Rintala et al [18] also examined the intestinal microbiota of infants at-risk of CD using 16S rRNA sequencing at 9 and 12 months of age in 9 subjects that developed CD by age four and 18 matched controls; no significant differences in microbiota composition were identified. Finally, Huang et al [19] examined intestinal microbiota using 16S rRNA sequencing at ages 1, 2.5, and 5 years in 16 subjects with CD (11 of whom developed CD after age 5) and 16 controls and found significant differences in the taxonomic composition in cases compared to controls at all time points.

While these studies have provided important foundations concerning alterations in the gut microbiota of subjects at risk of CD early in life, they have analyzed only up to three time points in the first year after birth [17-19] or otherwise have been restricted to only one subject with CD [15]. In addition, these studies used 16S rRNA sequencing to analyze the intestinal microbiota, which cannot provide insights into the functional characteristics of the microbiota nor can it provide any taxonomic information at the strain level both of which are necessary for designing effective treatments for CD. Furthermore, metabolomics analysis (if any) in these studies was often limited to serum (as opposed to fecal) metabolites, which do not provide any direct information about the metabolic activity of the gut microbiota. To gain insight into the pathogenesis of CD and other autoimmune diseases, prospective longitudinal studies are needed that prospectively examine subjects at multiple time points before the disease development [20].

Described herein is a prospective cohort study for CD called the Celiac Disease Genomic, Environmental, Microbiome and Metabolome study (CDGEMM) [21], which has been following nearly 500 infants in the US, Italy and Spain, with a first-degree relative with CD who are thus at a high risk of developing CD. Provided herein are inter-subject and intra-subject analyses using fecal metagenomic and metabolomic data collected at multiple time points before the onset of CD in 10 cases and 10 matched controls that identify alternations in the intestinal microbiome and metabolome before the onset of CD.

This provides an opportunity to observe alterations in the gut microbiota in the earliest steps of CD development because each subject serves as their own control. A similar approach has been pursued fairly recently for the study of the gut microbiota in infants at risk of T1D [1] in the first five years after birth. T1D is a complex chronic immune condition that peaks between 10-14 years of age and additionally its environmental trigger is unknown [2]. CD, on the other hand, is the only autoimmune disorder for which the trigger is known. Furthermore, previous prospective birth cohort studies, which did not examine the microbiome but did monitor at-risk infants during a 10-year period found that 80% of infants that developed CD did so by 36 months of age [3, 4]. This is unlike other autoimmune conditions such as T1D [2], IBD [5], or multiple sclerosis [6], for which typical onset is adolescence or adulthood. Despite the differing time lines, CD can serve as an ideal model of chronic immune-based disorders and can be studied prospectively to identify key steps, including microbiota surveillance, involved in the loss of tolerance to gluten.

Presented herein are the results of a cross-sectional analysis of cases compared to matched controls at the time of CD onset. We found that cases had a lower abundance of a number of microbes including Bacteroides vulgatus str_3775_S_1080 Branch and Bacteroides uniformis_ATCC_8492 for which a decreased abundance has previously reported to be associated with T1D [7] and impaired immune function [8]respectively. However, the cross-sectional analysis did not identify any changes in pathways abundances. The longitudinal analysis of microbial species, pathways, and metabolites revealed additional layers of insight into major shifts in the gut microbiota and metabolome in cases before CD onset. Notably, multiple microbial species, pathways, and metabolites were identified as having increased abundance before CD onset that have been previously linked to inflammatory and autoimmune conditions, others with decreased abundance before CD onset that have been reported to have anti-inflammatory properties, and several others that have not been reported before and are presumably CD specific. For example, there was an increased abundance of Dialister invisus, Parabacteroides sp., and Lachnospiraceae bacterium in cases, which have been previously been linked to pre-T1D [9], Behcet's disease [10], and obesity [11], respectively. There was also a decrease in abundance of a number of species and strains such as Streptococcus thermophilus, Faecalibacterium prausnitzii, and Clostridium clostridioforme in cases, which have been previously linked to acting as a probiotic [12], blocking the release of inflammatory cytokines [13, 14], and butyrate production [15]. In addition, novel microbes with increased abundance were identified in cases that have not been reported before and are thus likely to be CD-specific. A notable example is Porphyromonas sp., specifically strain 31_2, for which we observed an increase in abundance at all time points except −18 months. Other species of Porphyromonas such as Porphyromonas gingivalis has previously been reported to lead to the activation of the innate immune system [16] and impairment of the gut barrier [17, 18] and to contribute to the development of rheumatoid arthritis (RA) and increased disease severity [16]. The persistent increase in abundance of Porphyromonas sp in cases prior to CD onset thus suggests that Porphyromonas sp. 31_2 is a novel strain that contributes to CD pathogenesis. These novel microbes highlight the importance of taking into account for the role of species- and strain-level differences when studying different diseases.

The present longitudinal analysis of microbial species, pathways, and metabolites in controls also revealed alterations in the microbes and metabolites in control subjects linked to protection against chronic immune-based disorders. For example, control subjects were found to have an increase in abundance of Eubacterium eligens, six strains of Bifidobacterium longum, and nine strains of Bifidobacterium breve. An increase in abundance of B. longum has been reported to be anti-inflammatory in animal models of gliadin induced enteropathy [19, 20]. Furthermore, previous work found a higher abundance of B. longum in control subjects in the first 6 months after birth compared to subjects that later developed CD [21]. B. Breve has previously been shown to reduce the production of serum pro-inflammatory cytokines when administered to newly diagnosed patients with CD [22] possibly induced by the production of short chain fatty acids [23]. Pathway analysis revealed only minimal changes in pathways in controls including a decreased abundance in sulfur metabolism for which an increase has been associated with T1D [24]. Finally, metabolomic analysis identified several metabolites of significance not previously associated with protection against autoimmune conditions.

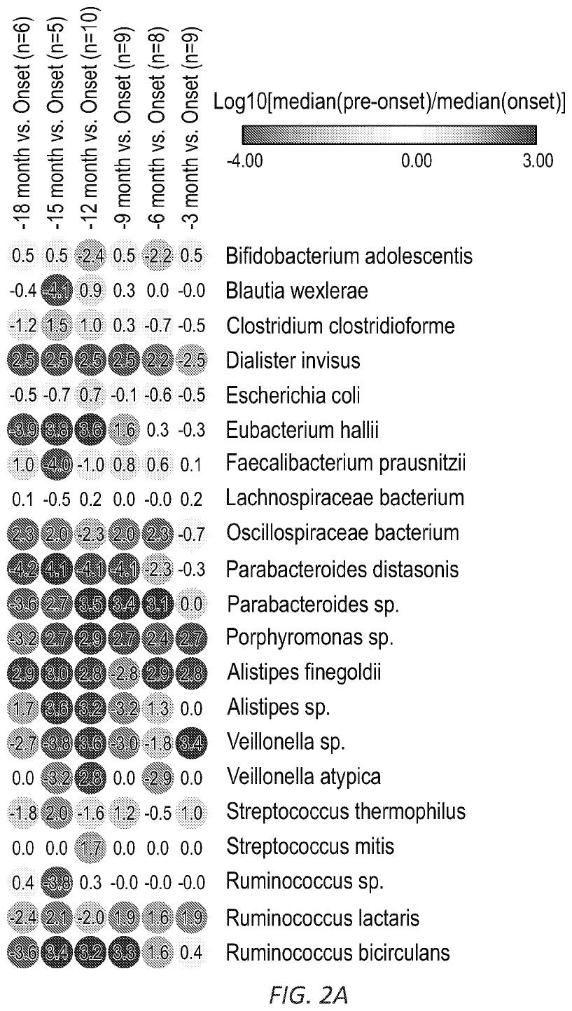

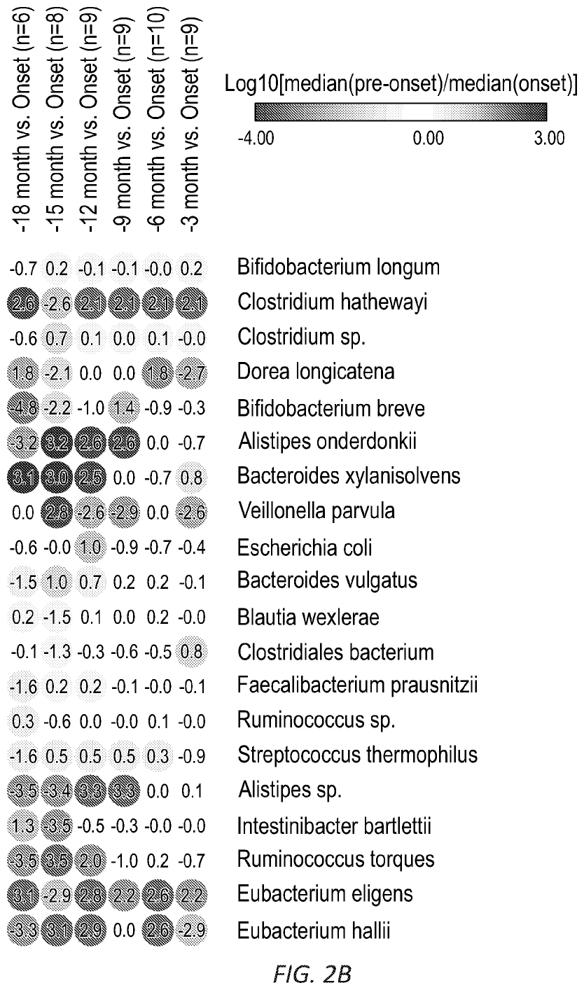

The present study further assessed whether there are any connections between identified microbes, pathways or metabolites with altered abundances before CD onset by performing an association study (using the Pearson correlation coefficient) between microbes and pathways or metabolites For example, in cases there was a positive association at −15 months between Bifidobacterium adolescentis and high-mannose type N-glycan biosynthesis, both of which increased in abundance at several time points before CD onset in our analysis (FIGS. 2A-B). As noted earlier, N-glycan has been associated with a number of autoimmune conditions [25-28] and additionally B. adolescentis has been previously described as increased in subjects with CD [29], This implies that B. adolescentis is involved in increased risk of developing autoimmunity and CD because either it contributes directly to high-mannose type N-glycan biosynthesis in the gut or it positively interacts with other microbes that are responsible for this pathway. The association analysis of microbes and metabolites also identified a positive association between Lachnospiraceae bacterium and the metabolite serine at all time points from −15 months to CD onset in cases. There was an increased abundance of Lachnospiraceae species in cases at all time points except −6 and −15 months compared to CD onset. Additionally, Lachnospiraceae and serine are associated with inflammatory conditions [11] and regulation of the adaptive immune response [30], respectively. This suggests that Lachnospiraceae increases the risk of developing autoimmune and inflammatory conditions such as CD either by producing serine or by positively interacting with other gut microbe that produce serine. Other interesting examples include a positive association in cases between Clostridium clostridioforme (whose abundance decreased in cases at several time points before CD onset) and the metabolite pinitol, which has been reported to have anti-inflammatory properties [31] and a positive association between Bifidobacterium longum, (specifically subsp longum ATCC 55813), which was increased in controls at all time points except −18 months, and the metabolite N-acetyl-D-galactosamine, which has been reported to inhibit the expression of the pro-inflammatory cytokine TNG-a [32].

A variety of chronic inflammatory diseases, including autoimmune disorders, aging, metabolic disorders, cancer, and neuro-degenerative disorders, shared common features. Specifically, genetic predisposition, exposure to specific environmental triggers, loss of barrier function that protect against uncontrolled antigen trafficking, a “hyperactive” immune system and a change in composition and function of the microbiome (particularly the gut microbiome), are the five pillars that dictate the balance between health and disease [33]. What is still not entirely clear is how the environment may epigenetically affect the expression of our genes by shifting genetic predisposition to a clinical outcome. This shortfall is due to the many variables at play that limit our capability to properly pinpoint to specific factors. Celiac disease offers a unique opportunity in minimizing these variables, since it is the only chronic inflammatory condition in which a specific genetic signature and the environmental trigger are well known. Based on the present studies, it is becoming clear that the epigenetic pressure of the environment has as universal transductor the composition and function of the microbiome, which seems to be responsible for dictating progression from pre-clinical genetic predisposition to clinical outcome. Therefore, the microbiome findings based on longitudinal analysis described herein, which revealed a series of strains that can exert a protective (anti-inflammatory) or deleterious (pro-inflammatory) effect, can be extrapolated to a variety of conditions including Type 1 Diabetes (T1D), Type 2 diabetes, multiple sclerosis, rheumatoid arthritis, Alzheimer's, Schizophrenia, asthma, obesity, food allergies, and inflammatory bowel diseases. Thus the present findings can be applied to a multitude of chronic inflammatory diseases.

### Methods of Diagnosis and Prediction

Included herein are methods for diagnosing and predicting risk of developing a chronic inflammatory disease. The methods rely on detection of specific bacterial strains or metabolites as described herein that are characteristic of inflammation and disease susceptibility.

The methods can include obtaining a sample from a subject, e.g., a fecal sample, and detecting in the sample the presence and/or level of one or more pro-inflammatory biomarkers, e.g., bacterial species, e.g., specific pro-inflammatory bacterial strains as listed in Table A, and/or specific pro-inflammatory metabolites as listed in Table C. The methods can also include detecting in the sample the presence and/or level of one or more anti-inflammatory biomarkers, e.g., bacterial species, e.g., specific anti-inflammatory bacterial strains as listed in Table B, and/or specific anti-inflammatory metabolites as listed in Table D.

The methods can include comparing the presence and/or level of the biomarkers with one or more references, e.g., a control reference that represents a normal level of the biomarker, e.g., a level in an unaffected subject or a subject who is not likely to develop a chronic inflammatory disease within the next 6, 12, 18, or 24 months, and/or a disease reference that represents a level of the biomarkers associated with the presence or likelihood of developing a chronic inflammatory disease within the next 6, 12, 18, or 24 months. Suitable reference values can include those shown in the figures.

Methods for detecting the presence of specific pro-inflammatory bacterial strains can include optionally isolating or purifying a biomarker, and determining its identity and/or quantity.

Various methods are well known within the art for the identification and/or isolation and/or purification of a biological marker from a sample. An “isolated” or “purified” biological marker is substantially free of cellular material or other contaminants from the cell or tissue source from which the biological marker is derived, i.e., partially or completely altered or removed from the natural state through human intervention. For example, nucleic acids contained in the sample can first be isolated according to standard methods, for example using lytic enzymes, chemical solutions, or isolated by nucleic acid-binding resins following the manufacturer's instructions.

The presence and/or level of a bacterial species, e.g., a specific pro-inflammatory bacterial strain as listed in Table A, or an anti-inflammatory strain as listed in Table B, can be determined by detecting one or more proteins and/or nucleic acids that are unique to or identify the strain. In some embodiments, the methods include extraction of prokaryotic DNA, e.g., genomic DNA, from stool samples; Shotgun metagenomic sequence (e.g., using pyrosequencing) of the microbiota DNA; and comparison of the obtained sequences to publicly available sequences, e.g., libraries such as GenBank, for strain identification and assignment. See, e.g., Wooley et al., PLoS Comput Biol. 2010 Feb; 6(2): e1000667. Probes/primers specific for each of the strains are also known in the art.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e., physical linkage) of a detectable substance, such as a radioactive agent or fluorophore (e.g., phycoerythrin (PE) or indocyanine (Cy5)), to an antibody or probe, as well as indirect labeling of the probe or antibody (e.g., horseradish peroxidase (HRP)) by reactivity with a detectable substance.

In some embodiments, an ELISA method may be used, wherein a surface such as the wells of a mictrotiter plate is coated with an antibody against which the protein is to be tested. The sample containing or suspected of containing the biological marker is then applied to the surface. After a sufficient amount of time, during which antibody-antigen complexes would have formed, the surface is washed to remove any unbound moieties, and a detectably labelled molecule is added. Again, after a sufficient period of incubation, the surface is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559) ; RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of a biomarker. Measurement of the level of a biomarker can be direct or indirect. For example, the abundance levels of a biomarker can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as 18S ribosomal RNA; Actin, beta; lyceraldehyde-3-phosphate; dehydrogenase; Phosphoglycerate kinase 1; Peptidylprolyl isomerase A; Ribosomal protein L13a; Ribosomal protein,; large, P0; Acidic ribosomal; phosphoprotein PO; Beta-2-microglobulin; Tyrosine; 3-monooxygenase/tryptophan5-monooxygenase activation protein, zeta polypeptide; Succinate dehydrogenase; complex, subunit A, flavoprotein (Fp); Transferrin receptor; Glucuronidase, beta; Hydroxymethylbilane; synthase; Hypoxanthine; phosphoribosyltransferase 1; and TATA box binding protein, are commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the presence and/or level of pro-inflammatory biomarkers is comparable to the presence and/or level of the biomarker(s) in the disease reference, and the subject has one or more symptoms associated with an inflammatory disease, then the subject can be diagnosed with the disease. In some embodiments, the subject has no overt signs or symptoms of an inflammatory disease, but the presence and/or level of one or more of the proteins evaluated is comparable to the presence and/or level of the protein(s) in the disease reference, then the subject has asymptomatic disease or an increased risk of developing an inflammatory disease. In some embodiments, once it has been determined that a person has an inflammatory disease, or has an increased risk of developing an inflammatory disease, then a treatment, e.g., as known in the art or as described herein, can be administered. For example, when a subject is diagnosed as having, or as having an increased risk of developing, celiac disease, the methods can include instructing the subject to adopt a gluten-free diet, and/or administering a treatment as described herein.

Suitable reference values can be determined using methods known in the art, e.g., using standard clinical trial methodology and statistical analysis. The reference values can have any relevant form. In some cases, the reference comprises a predetermined value for a meaningful level of a biomarker, e.g., a control reference level that represents a normal level of the biomarker, e.g., a level in an unaffected subject or a subject who is not at risk of developing a disease described herein, and/or a disease reference that represents a level of the proteins associated with conditions associated with chronic inflammation, e.g., a level in a subject having chronic inflammation (e.g., celiac disease).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. In some embodiments, an “increased risk” means that the subject has a level that is associated with (or statistically similar to) a level in a subject who was later determined to develop an inflammatory disease, e.g., within the subsequent 6, 12, 18, or 24 months.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g. CD), but is a first degree relative of a subject who has the disorder (e.g., a parent, child, or sibling). In some cases it may be desirable that the control subject has CD and in other cases it may be desirable that a control subject does not have CD.

A disease reference subject is one who has developed (or who later develops, when longitudinal data is used) a chronic inflammatory condition. An increased risk is defined as a risk above the risk of subjects in the general population, or above the risk of subjects who have a genetic risk factor for the condition, e.g., who have a first degree relative who has the disorder (e.g., a parent, child, or sibling).

Thus, in some cases the level of a pro-inflammatory biomarker in a subject being less than or equal to a reference level of a pro-inflammatory biomarker is indicative of a clinical status (e.g., indicative of the absence of the disorder or lower risk of as described herein). In other cases the level of a biomarker in a subject being greater than or equal to the reference level of an pro-inflammatory biomarker is indicative of the presence of disease or increased risk of the disease. Additionally, in some cases the level of an anti-inflammatory biomarker in a subject being greater than or equal to a reference level of an anti-inflammatory biomarker is indicative of a clinical status (e.g., lower risk of as described herein). In other cases the level of an anti-inflammatory biomarker in a subject being less than or equal to the reference level of an anti-inflammatory biomarker is indicative of the presence of disease or increased risk of the disease. In some embodiments, the amount by which the level in the subject is the less than the reference level is sufficient to distinguish a subject from a control subject, and optionally is a statistically significantly less than the level in a control subject. In cases where the level of a biomarker in a subject being equal to the reference level of the biomarker, “being equal” refers to being approximately equal (e.g., not statistically different).

The predetermined value can depend upon the particular population of subjects (e.g., human subjects) selected. For example, an apparently healthy population will have a different ‘normal’ range of levels of a biomarker than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

### Methods of Treatment

The methods described herein include methods for the treatment of chronic inflammatory conditions. In some embodiments, the disorder is celiac disease, T1D, IBD, food allergy (e.g., gluten or lactose intolerance), and irritable bowel syndrome (IBS). Generally, the methods include administering a therapeutically effective amount of an anti-inflammatory strain or metabolite as described herein, to a subject who is in need of, or who has been determined to be in need of, such treatment.

As used in this context, to “treat” means to ameliorate at least one symptom of the disorder associated with chronic inflammation, e.g., chronic gut inflammation. For example, CD is associated with intolerance to gluten and ingestion of gluten can cause abnormal gut function including nutrient malabsorption/malnutrition, bloating, diarrhea, constipation, weight loss, iron-deficiency anemia, fatigue, “brain fog,” depression, dermatitis herpetiformis, and other symptoms (See, e.g., Faulkner-Hogg et al., Scand J Gastroenterol. 1999 Aug;34(8):784-9; Zipser et al., Dig Dis Sci. 2003 Apr;48(4):761-4; Ferretti et al., Nutrients. 2012 Apr; 4(4): 243-257). Thus, a treatment as described herein can result in a reduction in any one or more of these symptoms intolerance, or a reduction in risk of developing CD and a return or approach to normal gut function. Administration of a therapeutically effective amount of a compound described herein for the treatment of a condition associated with chronic inflammation will result in decreased inflammation. In subjects who are at risk of developing, but who have not yet developed, a chronic inflammatory disease such as CD, the methods can reduce risk of developing the disease, or delay progression or development of the disease.

The present methods can be used to treat any subject, e.g., who is at risk, e.g., based on family history/genetic testing, or who is identified as at increased risk using a method described herein.

The present methods include the administration of the specific species and strains and metabolites, e.g., those that are depleted in subjects, e.g., children, who have or are at risk of developing a chronic inflammatory disease, e.g., celiac disease, to reduce inflammation, and maintain tolerance to gluten and therefore treat or prevent (reduce the risk of) onset of the disease.

The methods can include administering one or more of the gut microbiota species and strains listed in Table B, or the metabolites listed in Table D, that were identified herein as protective. Administration to patients with celiac disease can be used to treat the disease, or in children or adults at risk of celiac disease to prevent it.

### Pharmaceutical Compositions and Methods of Administration

The methods described herein include the use of pharmaceutical compositions comprising one or more gut microbiota species and strains listed in Table B, or one or more of the metabolites listed in Table D, as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use (e.g., comprising a metabolite) can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like; however in compositions comprising therapeutic microorganisms antibacterial agents should not be present. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, optionally followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for administration as a liquid or drink. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

Pharmaceutical compositions comprising anti-inflammatory microbes as described herein may optionally comprise one or more possible pharmaceutically acceptable excipients, such as carriers, preservatives, cryoprotectants (e.g., sucrose and trehalose), stabilizers, adjuvants, and other substances. For example, when the composition comprises anti-inflammatory microbes that are alive, excipients are chosen such that the live bacterium is not killed, or such that the ability of the bacteria to effectively colonize a subject is not compromised by the use of excipients. Suitable pharmaceutical carriers are known in the art and, for example, include liquid carriers, such as normal saline and other non-toxic salts at or near physiological concentrations, and solid carriers, such as talc and sucrose. In some embodiments, the pharmaceutical composition comprises an adjuvant. In some embodiments, the pharmaceutical composition may be a in a form suitable for aerosolized administration to a subject. In some embodiments, the pharmaceutical formulation is in a freeze-dried form (i.e., lyophilized form). In some embodiments, the pharmaceutical formulation is a gelatin capsule. Suitable pharmaceutical carriers and adjuvants and the preparation of dosage forms are described in, Remington's Pharmaceutical Sciences, 17th Edition, (Gennaro, Ed., Mack Publishing Co., Easton, Pa., 1985), which is herein incorporated by reference.

Administration of the anti-inflammatory microbes described herein to a subject can be by any known technique, including, but not limited to oral administration, rectal administration, vaginal administration, or nasal administration.

The dose of the anti-inflammatory microbes that is administered to a subject can and will vary depending on the anti-inflammatory microbes selected, the route of administration, and the intended subject, as will be appreciated by one of skill in the art. Generally speaking, the dosage need only be sufficient to elicit a protective host response in the subject. For example, typical dosages for oral administration could be about 1×10⁷ to 1×10¹⁰ colony forming units (CFU) depending upon the age of the subject to whom the bacteria will be administered. Administering multiple dosages of the anti-inflammatory microbes may also be used as needed to provide the desired level of protection.

Kits comprising anti-inflammatory microbes or a pharmaceutical composition described herein are also provided. In some embodiments, the kit further comprises instructions for use. In some embodiments, the pharmaceutical composition is lyophilized such that addition of a hydrating agent (e.g., buffered saline) reconstitutes the composition to generate a pharmaceutical composition suitable for administration to a subject (e.g., orally).

The pharmaceutical compositions can be included in a container, pack, or dispenser together with instructions for administration.

### EXAMPLES

The invention is further described in the following examples, which do not limit the scope of the invention described in the claims.

### Methods

The following materials and methods were used in the Examples below.

### Study population

Ten infants who developed CD and their matched controls were selected from the CDGEMM prospective birth cohort study [34]. CDGEMM enrolled healthy infants between the ages of 0-6 months who have a first-degree relative with CD and follows them prospectively for ten years. As part of the study, questionnaires were sent to the parents at enrollment to obtain information related to the infants' environment at birth and at delivery as well as monthly diaries to monitor food intake and antibiotic exposure. In addition to clinical information, stool samples were collected into 4 tubes every three months from birth to three years of age and then every six months until five years. Two tubes of stool were stored to allow for processing for both metagenomic and metabolomic analysis. Serum was collected every 6 months until 3 years of age and then yearly until 5 years of age. All infants underwent serum testing for antibodies to IgA tTG and IgG deamidated gliadin peptide (dGP) using QUANTA Lite Rh-tTG IgA ELISA (INOVA Diagnostics, San Diego, Calif., USA) on the BioFlash platform at each collection. HLA genetic type was determined from whole blood collected at 12 months of age using the DQ-CD Typing Plus (BioDiagne, Palermo, Italy) per the manufacturer's instructions.

Infants found to have IgA tTG levels above the kit reference value (>20 CU) were subjected to confirmatory testing for IgA endomysial antibodies (EMA) using the NOVA Lite Monkey Oesophagus IFA Kit (Inova Diagnostics, San Diego, Calif., United States). Furthermore, infants found to have elevated IgG dGP in the absence of elevated IgA tTG were evaluated for potential IgA deficiency. A total IgA level measurement was performed for serum samples from these individuals using immunoturbidimetric methods (LabCorp, Burlington, N.C., United States). Parents were informed of serology results after each blood draw, and if positive instructed to follow up with their physician for further confirmatory testing including repeating blood work and endoscopy. Written informed consent was obtained from the parents of infants included in the study according to the standards outlined and approved by the Partners Human Research Committee Institutional Review Board.

### DNA Extraction

Stool samples for metagenomic analysis were stored and processed in the United States. Total DNA from each sample was extracted using the Qiagen Power soil DNA extraction kit (Qiagen, Hilden, Germany).

### Metagenomic Sequencing

Isolated DNA was quantified by Qubit 2.0 (ThermoFisher, USA). DNA libraries were prepared using the Illumina Nextera XT library preparation kit according to the manufacturer's protocol. Library quantity and quality was assessed with Qubit (ThermoFisher, USA) and Tapestation (Agilent Technologies, Calif., USA). Libraries were then sequenced on Illumina HiSeq 400 platform on 2×150 bp run at CosmosID Inc.(Rockville, Md., USA).

### Taxonomic Profiling

Taxonomic profiling of metagenomics samples was performed at both species- and strain-level resolution using the commercial CosmosID metagenomic analysis platform (CosmosID Inc., Rockville, Md., USA) as described elsewhere [35, 36].

### Functional Profiling

Functional profiling to identify functional pathways encoded by each metagenome and their abudnaces was performed as follows: Raw metagenomic reads were first trimmed using BBduk (jgi.doe.gov/data-and-tools/bbtools/) (using minlen=25). Next, we used Spades [37] to perform metagenome assembly for each sample followed by Prodigal [38] to identify coding sequences (CDSs) in the assembled metagenomes and Interproscan [38] to assign biochemical functions to identified genes/proteins based on KEGG pathways. The relative abundance of each gene was computed using

$$G = \frac{L * C}{( R - K + 1 )} ,$$

where, G is fragments per kilobase of gene per million (FPKM), L is the length of the gene, C is the coverage of the contig on which the gene is identified, R is the read length and K is the Kmer size [39]. The relative abundances of each KEGG pathway was then quantified by summing the relative abundances of all the genes associated to that pathway.

### Metabolomic Profiling

All stool samples for metabolomics were stored and processed in Italy. The metabolome extraction, purification and derivatization were carried by the MetaboPrep GC kit (Theoreo, Montecorvino Pugliano, Italy) according to manufacturer instructions. Instrumental analyses were performed with a GC-MS system (GC-2010 Plus gas chromatograph and QP2010 Plus mass spectrometer;

Shimadzu Corp., Kyoto, Japan). Sample analysis was performed in triplicate. The molecular identity of metabolites was determined by analysis of the corresponding mass spectrum in the chromatogram, setting the linear index difference max tolerance to 10. These identified metabolites were further confirmed using external standards according to level 1 Metabolomics Standards Initiative (MSI) [40].

### Cross-Sectional and Longitudinal Analyses

Cross-sectional analysis to identify species, strains, pathways or metabolites whose abundance is significantly different between cases and controls at a given time point (i.e., CD onset) was performed by using the Mann-Whitney U (Wilcoxon Rank-Sum) test (a p-value threshold of 0.05 was used to report significant results). Longitudinal differential abundance analysis of species, strains, pathways or metabolites between each time point before CD onset (−18, −15, 12, −9, −6 and −3 months) and the time of CD onset (t=0) was performed by using paired Wilcoxon (Wilcoxon Signed Rank) test points with the same p-value thresholds noted above to report the significant results. Any longitudinal pattern observed for both cases and controls was not reported. All analyses for species, strains and pathways were performed in Python (using “mannwhitneyu” and “Wilcoxon” functions of scipy.stats library) and those for metabolites were performed in R (using the “Ttest.Anal” function of MeteaboAnlyst package).

### Example 1. Prospective Longitudinal Birth Cohort Study Design

Ten children from CDGEMM that developed CD by the time of inception of the current study were evaluated (“cases”). We identified matched controls for each one of these cases according to HLA genetics and environmental exposures to focus on alterations in the gut microbiota related to CD. To this end, infants were matched, when possible, by season of birth, location of birth, sex, birth delivery mode, HLA genetics, timing of solid food and gluten introduction. Characteristics of these 10 cases and their 10 matched controls are presented in Table 1.

While we have been collecting fecal samples every 3 months for the first 3 years after birth and then every 6 months until age 10, in this study we focused on a time window spanning 18 months before the time of CD onset until the CD onset to ensure that very early changes in microbiome shift would be captured. Additionally, the youngest age at which CD was diagnosed in our cohort is 18 months, further supporting our choice in surveilling microbiome shifts at a very upstream timepoint.

For the purpose of our study, CD onset is defined as elevated serum anti-tissue transglutaminase (tTG) and anti-endomysial antibody (EMA) in our research laboratory after which subjects are referred for clinical confirmation of CD with additional blood testing and duodenal biopsies when indicated following the revised ESPGHAN criteria [41]. For our analysis, we took the time of CD onset, as our reference point (t=0) and converted all other sample collection times to relative time points with respect to the time of CD onset. This resulted in six relative time points (in addition to t=0) including −18 months, −15 months, −12 months, −9 months, −6 months and −3 months with the negative sign implying the time before the CD onset. A total of 118 fecal sample collected at these time points underwent shotgun metagenomic sequencing and metabolomic profiling. Taxonomic profiling of metagenomic samples was performed at both species- and strain-level resolution and functional profiling was also performed to identify functional pathways encoded by each metagenome (see Methods). The identified taxonomic, functional and metabolite profiles were then rigorously analyzed as described below to identify inter-subject and intra-subject variations in the gut microbiome.

### Example 2. Changes in the Microbiota of Cases Compared to Controls at the Time of CD Onset

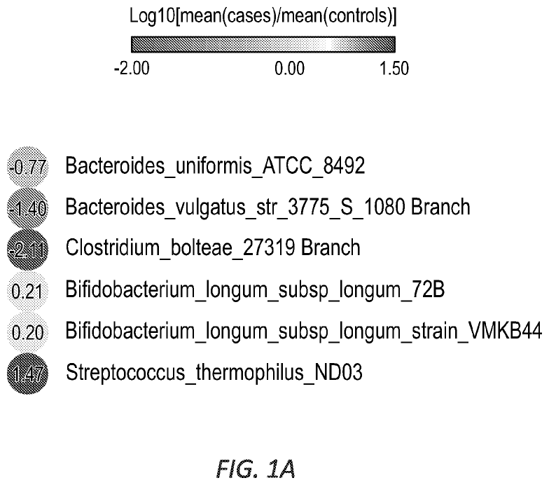

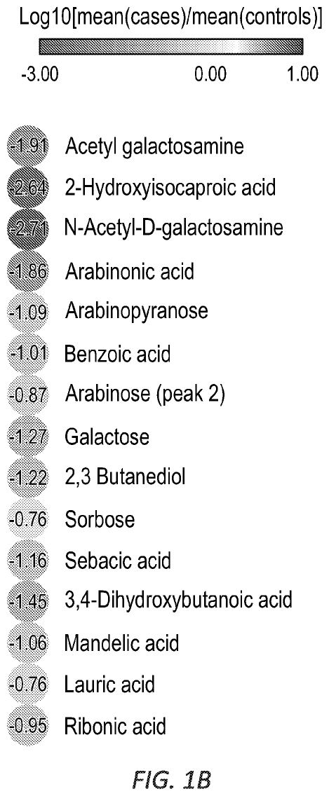

We performed a cross-sectional (inter-subject) analysis to explore various features of the gut microbiota (microbes, pathways and metabolites) differ between cases and controls once a subject develops CD (CD onset, FIGS. 1A-B). The analysis did not identify microbes at the species level that where significantly different in cases compared to controls at CD onset. The analysis did, however, identify a number of microbes at the strain level and metabolites whose abundances are significantly different between the two groups. For example, we found that cases have significantly less Bacteroides vulgatus str_3775_S_1080 Branch (FIG. 1). A decreased abundance of B. vulgatus has been reported to lead to an increased gut microbial production of lipopolysaccharide (LPS), which will impair immune function [42]. A decreased abundance of B. vulgatus has been also reported in infants who developed T1D compared to matched controls [7]. We found that cases also had a significantly decreased abundance of Bacteroides uniformis_ATCC_8492. Previous research in mice has shown that Bacteroides uniformis decreases TNF-α production and increases IL-10 production [8] resulting in improved immune defense mechanisms. Thus a decrease in this strain, as seen in cases, may be associated with a decrease in immune defense mechanisms. Despite several changes at taxonomic level, we did not identify any functional pathways whose abundance is significantly different between cases and controls at the time of CD onset. Cross-sectional analysis of metabolites identified several previously unreported metabolites with decreased abundance in cases compared to control subjects at CD onset such as acetyl galactosamine, 2-hydroxyisocaproic acid and arabinoic acid among others (FIG. 1B). The only identified metabolite implicated in autoimmunity based on existing literature is lauric acid whose abundance has decreased in cases compared to controls in our study. This is in contrast with previous reports that this metabolite has pro-inflammatory effects through promoting Th1 and Th17 differentiation in mice, which results in a more severe course of experimental autoimmune encephalitis [43].

### Example 3. Longitudinal Changes in the Microbiota of Cases and Controls

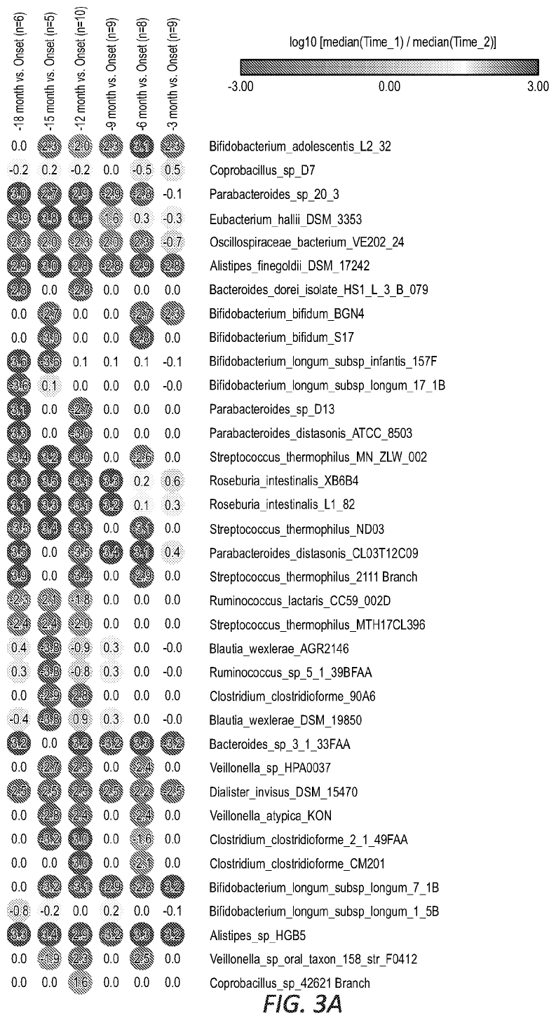

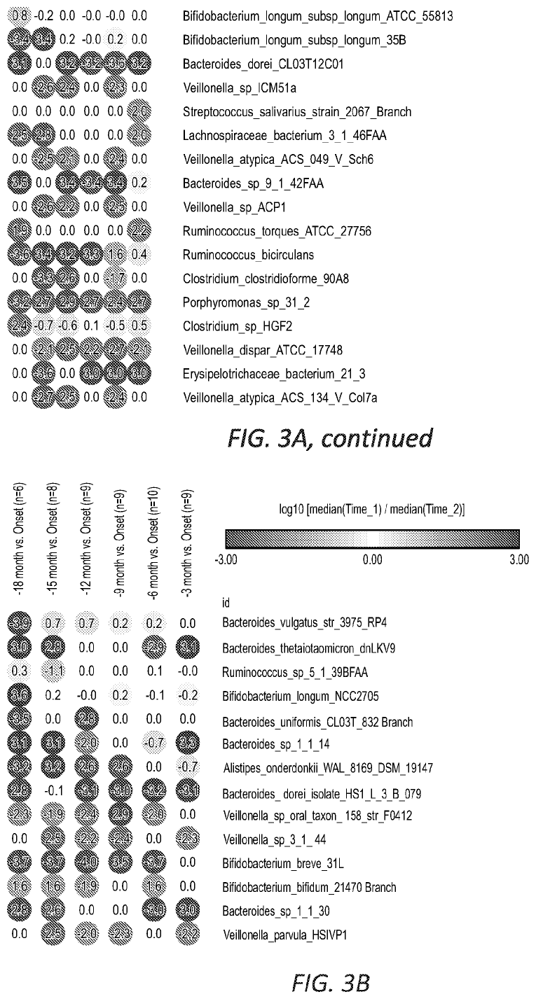

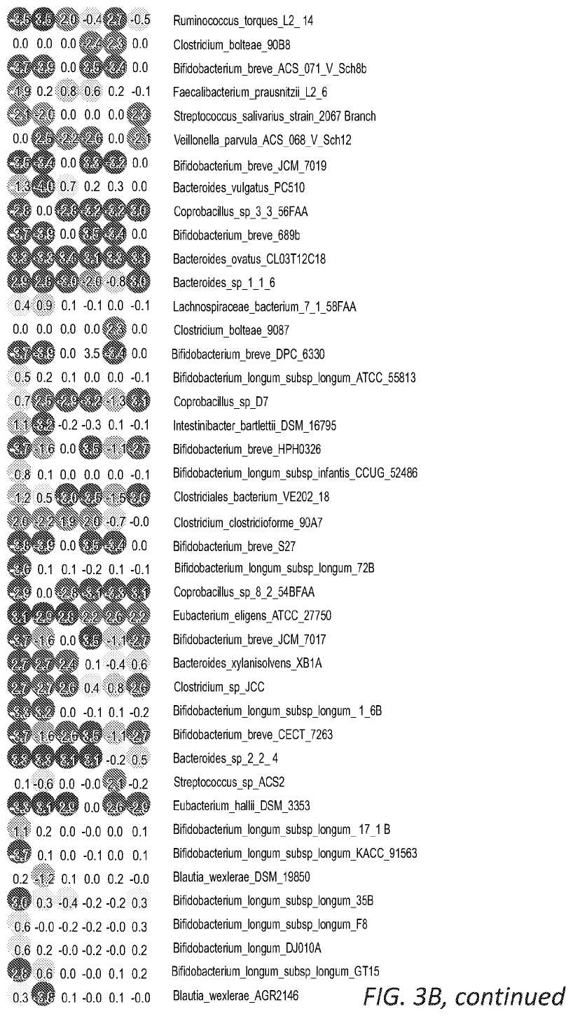

Due to the prospective longitudinal design of our birth cohort study, we were able to perform a longitudinal analysis to gain additional insights beyond a cross-sectional analysis by identifying intra-subject alterations in the gut microbiome before the onset of CD. Toward this end, we identified species, strains, pathways and metabolites whose abundance differentially change between a pre-onset time point (i.e., −18, −15, −12, −9, −6 and −3 months) and the CD onset (i.e., t=0). This analysis was performed for cases and controls separately and we report only non-overlapping longitudinal patterns between cases and controls (FIGS. 2-4). By longitudinal analysis of microbial species (FIGS. 2A-B), we found the increased abundance of a number of species previously associated with other autoimmune conditions in cases compared to CD onset, which may suggest that these microbes can serve as a biomarker of a future autoimmune disease. For example, our analysis identified a significantly higher abundance of Dialister invisus strain DSM_15470 at CD at all timepoints (except −3 months) compared to CD onset. An increased abundance of Dialister has been previously reported in children with pre-T1D compared to controls [9] and in subjects who later developed CD [44]. We also observed an increased abundance of Parabacteroides species and strains prior to CD onset in cases (FIGS. 2 and 3). Specifically, the longitudinal analysis found Parabacteroides sp. higher at all time points, except at −18 months, compared to CD onset. An increased abundance of Parabacteroides has previously been linked to autoimmune conditions such as T1D [45] and Behcet's disease [10]. However, for Parabacteroides distasonis we observed a decreased abundance at all pre-onset timepoints except for −15 months, Finally, Lachnospiraceae bacterium showed an increased abundance at all timepoints except for −15 and −6 months prior to CD onset. Lachnospiraceae bacterium colonization has been associated with obesity and diabetes in genetically at-risk mice [11] and has also been shown to induce colonic inflammation by recruiting macrophages into the colon in the presence of colonic epithelial cell disruption [46]. Table A presents a list of pro-inflammatory bacterial strains.

TABLE A
PRO-INFLAMMATORY MICROBES

```
Porphyromonas sp.
  Porphyromonas_sp_31_2
Ruminococcus bicirculans
  Ruminococcus_{—}bicirculans
Alistipes finegoldii
  Alistipes finegoldii_DSM_17242
Alistipes_sp_HGB5
Erysipelotrichaceae_bacterium_21_3
Dialister invisus
  Dialister_{—}invisus_DSM_15470
Veillonella parvula
  Veillonella_{—}parvula_ACS_068_V_Sch12
  Veillonella_{—}parvula_HSIVP1
Parabacteroides sp.
  Parabacteroides_sp_20_3
  Parabacteroides_sp_D13
Lachnospiraceae bacterium
  Lachnospiraceae_bacterium_3_1_46FAA
Bifidobacterium adolescentis
  Bifidobacterium_{—}adolescentis_L2_32
Parabacteroides distasonis
  Parabacteroides_{—}distasonis_ATCC_8503
  Parabacteroides_{—}distasonis_CL03T12C09
Clostridium sp.
  Clostridium_sp_JCC
Alistipes onderdonkii
  Alistipes_{—}onderdonkii_WAL_8169_DSM_19147
Other Strains:
  Streptococcus_{—}thermophilus_ND03
  Roseburia_{—}intestinalis_XB6B4
  Roseburia_{—}intestinalis_L1_82
  Veillonella_{—}dispar_ATCC_17748
  Veillonella_{—}atypica_ACS_134_V_Col7a
Bacteroides_{—}dorei_isolate_HS1_L_3_B_079
```

Our longitudinal analysis also revealed a number of species and strains previously identified as having anti-inflammatory properties whose abundance is lower during the “march” from pre-clinical to CD onset (FIGS. 2A-B and 3A-B).

For example, we observed a decreased abundance of Streptococcus thermophilus at −18, −12 and −6, however, the abundance is higher at all other time points. Streptococcus thermophilus has been identified as a probiotic, which releases an anti-inflammatory metabolite capable of crossing the intestinal barrier [12]. In addition, Faecalibacterium prausnitzii has a lower abundance at −15 and −12 months compared to CD onset though we observe a higher abundance at all other timepoints. F. prausnitzii is known to have anti-inflammatory properties through the release metabolites capable of blocking NF-κB activation and IL-8 production [13, 14] and has been reported to be under-abundant in subjects with IBD [47, 48]. We also found Clostridium clostridioforme, a microbe that contributes to butyrate production [15], to show a decreased abundance at all time points except for at −15 and −12 months compared to CD onset. A decreased abundance of C. clostridioforme in subjects with IBD has been reported before [49]. We also identified a number of previously unreported species and strains such as Ruminococcus lacti (−18 and −12 months), Blautia wexlerae (−15 months), Alisfipes finegoldii (−9 months) among others as significantly decreased compared to CD onset (FIGS. 2 and 3). Table B presents a list of anti-inflammatory bacterial strains.

TABLE B
ANTI-INFLAMMATORY MICROBES

```
Bacteroides_{—}ovatus_CL03T12C18
Clostridium_sp_JCC
Faecalibacterium prausnitzii
  Faecalibacterium_{—}prausnitzii_L2_6
Bifidobacterium longum
  Bifidobacterium_{—}longum_DJO10A
  Bifidobacterium_{—}longum_NCC2705
  Bifidobacterium_{—}longum_subsp_infantis_CCUG_52486
  Bifidobacterium_{—}longum_subsp_longum_1_6B
  Bifidobacterium_{—}longum_subsp_longum_17_1B
  Bifidobacterium_{—}longum_subsp_longum_35B
  Bifidobacterium_{—}longum_subsp_longum_72B
  Bifidobacterium_{—}longum_subsp_longum_ATCC_55813
  Bifidobacterium_{—}longum_subsp_longum_F8
  Bifidobacterium_{—}longum_subsp_longum_GT15
  Bifidobacterium_{—}longum_subsp_longum_KACC_91563
Bifidobacterium breve
  Bifidobacterium_{—}breve_31L
  Bifidobacterium_{—}breve_689b
  Bifidobacterium_{—}breve_ACS_071_V_Sch8b
  Bifidobacterium_{—}breve_CECT_7263
  Bifidobacterium_{—}breve_DPC_6330
  Bifidobacterium_{—}breve_HPH0326
  Bifidobacterium_{—}breve_JCM_7017
  Bifidobacterium_{—}breve_JCM_7019
  Bifidobacterium_{—}breve_S27
Eubacterium eligens
  Eubacterium_{—}eligens_ATCC_27750
Clostridium hathewayi
  No strain identified
  Bacteroides_{—}vulgatus_str_3775_S_1080 Branch
Blautia wexlerae
  Blautia_{—}wexlerae_AGR2146
  Blautia_{—}wexlerae_DSM_19850
Clostridium clostridioforme
  Clostridium_{—}clostridioforme_2_1_49FAA
  Clostridium_{—}clostridioforme_90A6
  Clostridium_{—}clostridioforme_90A8
  Clostridium_{—}clostridioforme_CM201
Ruminococcus lactaris
  Ruminococcus_{—}lactaris_CC59_002D
Alistipes finegoldii
  Alistipes_{—}finegoldii_DSM_17242
Bacteroides xylanisolvens
  Bacteroides_{—}xylanisolvens_XB1A
Clostridiales bacterium
  Clostridiales_{—}bacterium_VE202_18
Parabacteroides distasonis
  Parabacteroides_{—}distasonis_ATCC_8503
  Parabacteroides_{—}distasonis_CL03T12C09
Streptococcus thermophilus
  Streptococcus_{—}thermophilus_2111 Branch
  Streptococcus_{—}thermophilus_MN_ZLW_002
  Streptococcus_{—}thermophilus_MTH17CL396
  Streptococcus_{—}thermophilus_ND03
Ruminococcus sp.
  Ruminococcus_sp_5_1_39BFAA
Intestinibacter bartlettii
  Intestinibacter_{—}bartletti_DSM_16795
Eubacterium hallii
  Eubacterium_{—}hallii_DSM_3353
Other Strains:
  Clostridium_{—}bolteae_27319 Branch
  Bacteroides_{—}uniformis_ATCC_892
```

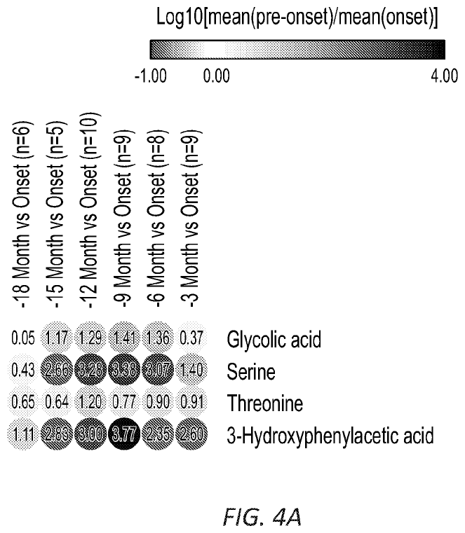

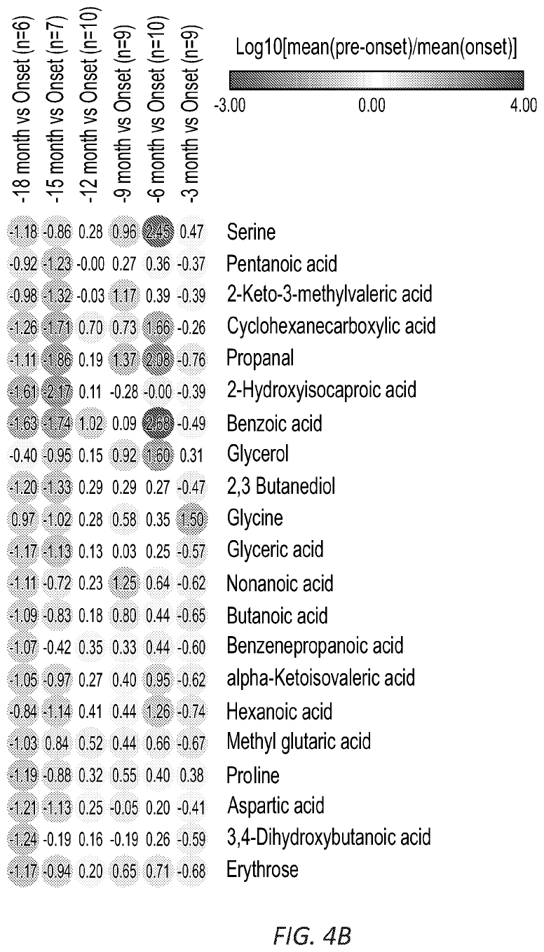

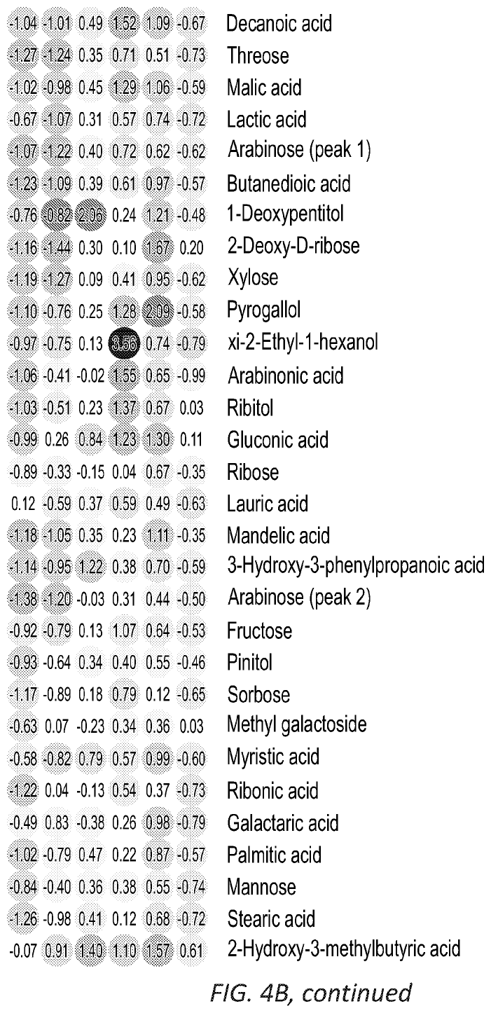

Longitudinal analysis of metabolites identified four metabolites in CD cases including glycolic acid, serine, threonine and 3-hydroxyphenylacetic acid, which were increased at all time points compared to CD onset (FIGS. 4A-B). Extracellular serine has been reported to regulate the adaptive immune response due to its essential role in stimulating effector T cell expansion [30]. Decreased threonine has previously been reported in the serum of patients with rheumatoid arthritis compared to controls, however, it is unclear how findings related to altered serum metabolites compares to altered fecal metabolites [50]. In addition, pre-treatment of PBMCs with microbially-derived 3-hydroxyphenylacetic acid has been shown to reduce inflammatory cytokine production when the PBMCs were stimulated by LPS suggesting this metabolite may have an anti-inflammatory role [51]. Tables C and D present pro- and anti-inflammatory metabolites, respectively.

TABLE C
PRO-INFLAMMATORY METABOLITES

Serine
Threonine
Glycolic acid

TABLE D
ANTI-INFLAMMATORY METABOLITES

2-Hydroxy-3-methylbutyric acid
Acetyl galactosamine
2-Hydroxyisocaproic acid
Arabinonic acid
Lauric acid
3-Hydroxyphenylacetic acid
Ribitol
Gluconic acid
Proline
Glycine
Glycerol
Serine

Our longitudinal analysis also revealed that some species and strains such as Bifidobacterium longum, Bacteriodes xylanisolvens and Clostridiales bacterium increased in abundance at −3 months and a number of other time points compared to t=0 in control subjects (CD onset in matched cases). In particular our analysis identified six strains of B. longum that were significantly increased in healthy controls at −3 months compared to t=0. These findings are in agreement with previous work, which found an increased abundance of B. longum in control subjects compared to subjects that later developed CD [21]. B. longum has also been shown to increase IL-10 production and decrease the production of inflammatory cytokines and the CD4+ T-cell immune response in animal models with gliadin induced enteropathy [19, 20] further supporting its possible role in the protection against chronic immune conditions. We also identified an increased abundance of Bifidobacterium breve and nine of its specific strains at −9 months compared to t=0. B. breve is a commonly used probiotic in infants, which has been linked to protection against necrotizing enterocolitis, the development of allergic diseases [52], and decreased inflammation in CD [22, 23]. We also found that control subjects have a significantly increased abundance of Escherichia coli at −12 months compared to t=0 even though the abundance at all other time points is decreased. E coli has been shown to stimulate B regulatory cells to produce anti-inflammatory cytokines and promote the development of T regulatory cells among other mechanisms to mitigate an inflammatory response [53].

We also observed an increase in the abundance of Clostridium hathewayi and Eubacterium eligens at all time points in controls compared to t=0 except for −15 months. E. eligens has been shown to promote the production of anti-inflammatory cytokines in vitro [54]. Additionally, we identified Veillonella parvula, a microbe associated with the autoimmune condition relapsing polychondritis [55], significantly decreased in abundance at −12, −9, and −3 months compared to t=0.

In contrast to our findings in cases, where we observed a significant change for the majority of pathways at −15 months, our longitudinal analysis of microbiome-encoded functional pathways in control subjects shows that the statistically significant change in the abundance of pathways occurs at −3 months even though the observed fold change is minimal. Only benzoate degradation via hydroxylation and ascorbate and aldarate metabolism, at −15 months compared to t=0 was notably increased in abundance. However, ascorbate and aldarate metabolism was decreased at all other time points compared to t=0. Utilizing RNAseq, we previously found ascorbate and aldarate metabolism to be downregulated in the intestinal mucosa of subjects with active CD compared to subjects in remission [56]. Finally, our analysis identified a decreased abundance sulfur metabolism (at all timepoints except −18) and LPS biosynthesis (at all timepoints except −18 and −15) for which an increase has been associated with T1D [24] and autoimmune hepatitis [57], respectively.

Our longitudinal metabolomic analysis in control subjects identified hydroxyl fatty acid 2-hydroxy-3-methylbutyric acid whose abundance increased at all timepoints compared to CD onset. We also identified several other metabolites with a decreased abundance at −18, −15, and −3 months and an increased abundance at −12, −9, and −6 months compared to t=0 in controls (FIGS. 4A-B).

### REFERENCES

1. Vatanen, T., et al., The human gut microbiome in early-onset type 1 diabetes from the TEDDY study. Nature, 2018. 562(7728): p. 589-594.

2. DiMeglio, L. A., C. Evans-Molina, and R. A. Oram, Type 1 diabetes. Lancet, 2018. 391(10138): p. 2449-2462.

3. Lionetti, E., et al., Introduction of gluten, HLA status, and the risk of celiac disease in children. N Engl J Med, 2014. 371(14): p. 1295-303.

4. Vriezinga, S. L., et al., Randomized feeding intervention in infants at high risk for celiac disease. N Engl J Med, 2014. 371(14): p. 1304-15.

5. Karlinger, K., et al., The epidemiology and the pathogenesis of inflammatory bowel disease. Eur J Radiol, 2000. 35(3): p. 154-67.

6. Thompson, A. J., et al., Multiple sclerosis. Lancet, 2018. 391(10130): p. 1622-1636.

7. Cinek, O., et al., Imbalance of bacteriome profiles within the Finnish Diabetes Prediction and Prevention study: Parallel use of 16S profiling and virome sequencing in stool samples from children with islet autoimmunity and matched controls. Pediatric diabetes, 2017. 18(7): p. 588-598.

8. Cano, P. G., et al., Bacteroides uniformis CECT 7771 ameliorates metabolic and immunological dysfunction in mice with high-fat-diet induced obesity. PloS one, 2012. 7(7): p. e41079.

9. Maffeis, C., et al., Association between intestinal permeability and faecal microbiota composition in Italian children with beta cell autoimmunity at risk for type 1 diabetes. Diabetes/metabolism research and reviews, 2016. 32(7): p. 700-709.

10. Ye, Z., et al., A metagenomic study of the gut microbiome in Behcet's disease. Microbiome, 2018. 6(1): p. 135.

11. Kameyama, K. and K. Itoh, Intestinal colonization by a Lachnospiraceae bacterium contributes to the development of diabetes in obese mice. Microbes and environments, 2014: p. ME14054.

12. Menard, S., et al., Lactic acid bacteria secrete metabolites retaining anti-inflammatory properties after intestinal transport. Gut, 2004. 53(6): p. 821-828.

13. Sokol, H., et al., Faecalibacterium prausnitzii is an anti-inflammatory commensal bacterium identified by gut microbiota analysis of Crohn disease patients. Proceedings of the National Academy of Sciences, 2008. 105(43): p. 16731-16736.

14. Quévrain, E., et al., Identification of an anti-inflammatory protein from Faecalibacterium prausnitzii, a commensal bacterium deficient in Crohn's disease. Gut, 2016. 65(3): p. 415-425.

15. Dehoux, P., et al., Comparative genomics of Clostridium bolteae and Clostridium clostridioforme reveals species-specific genomic properties and numerous putative antibiotic resistance determinants. BMC genomics, 2016. 17(1): p. 819.

16. Pouliot, M., et al., Lipoxin A4 analogues inhibit leukocyte recruitment to

Porphyromonas gingivalis: a role for cyclooxygenase-2 and lipoxins in periodontal disease. Biochemistry, 2000. 39(16): p. 4761-4768.

17. Sato, K., et al., Aggravation of collagen-induced arthritis by orally administered Porphyromonas gingivalis through modulation of the gut microbiota and gut immune system. Scientific reports, 2017. 7(1): p. 1-13.

18. Flak, M. B., et al., Inflammatory arthritis disrupts gut resolution mechanisms, promoting barrier breakdown by Porphyromonas gingivalis. JCI insight, 2019. 4(13).

19. Laparra, J. M., et al., Bifidobacterium longum CECT 7347 modulates immune responses in a gliadin-induced enteropathy animal model. PLoS One, 2012. 7(2).

20. Medina, M., et al., Bifidobacterium strains suppress in vitro the pro-inflammatory milieu triggered by the large intestinal microbiota of coeliac patients. Journal of Inflammation, 2008. 5(1): p. 19.

21. Olivares, M., et al., Gut microbiota trajectory in early life may predict development of celiac disease. Microbiome, 2018. 6(1): p. 36.

22. Klemenak, M., et al., Administration of Bifidobacterium breve decreases the production of TNF-α in children with celiac disease. Digestive diseases and sciences, 2015. 60(11): p. 3386-3392.

23. Primec, M., et al., Clinical intervention using Bifidobacterium strains in celiac disease children reveals novel microbial modulators of TNF-α and short-chain fatty acids. Clinical nutrition, 2019. 38(3): p. 1373-1381.

24. Brown, C. T., et al., Gut microbiome metagenomics analysis suggests a functional model for the development of autoimmunity for type 1 diabetes. PloS one, 2011. 6(10).

25. Chien, M. W., et al., The modulatory roles of N-glycans in T-cell-mediated autoimmune diseases. International journal of molecular sciences, 2018. 19(3): p. 780.

26. Yu, Z., et al., Family studies of type 1 diabetes reveal additive and epistatic effects between MGAT1 and three other polymorphisms. Genes & Immunity, 2014. 15(4): p. 218-223.

27. Dias, A. M., et al., Dysregulation of T cell receptor N-glycosylation: a molecular mechanism involved in ulcerative colitis. Human molecular genetics, 2014. 23(9): p. 2416-2427.

28. Mkhikian, H., et al., Genetics and the environment converge to dysregulate N-glycosylation in multiple sclerosis. Nature communications, 2011. 2(1): p. 1-13.

29. Sanchez, E., et al., Intestinal Bacteroides species associated with coeliac disease. Journal of clinical pathology, 2010. 63(12): p. 1105-1111.

30. Ma, E. H., et al., Serine is an essential metabolite for effector T cell expansion. Cell metabolism, 2017. 25(2): p. 345-357.

31. Singh, R., et al., Anti-inflammatory effect of (+)-pinitol. Fitoterapia, 2001. 72(2): p. 168-170.

32. Murakami, Y., et al., N-acetyl-d-galactosamine inhibits TNF-α gene expression induced in mouse peritoneal macrophages by fimbriae of Porphyromonas (Bacteroides) gingivalis, an oral anaerobe. Biochemical and biophysical research communications, 1993. 192(2): p. 826-832.

33. Furman, D., et al., Chronic inflammation in the etiology of disease across the life span. Nat Med, 2019. 25(12): p. 1822-1832.

34. Leonard, M. M., et al., Celiac Disease Genomic, Environmental, Microbiome, and Metabolomic (CDGEMM) Study Design: Approach to the Future of Personalized Prevention of Celiac Disease. Nutrients, 2015. 7(11): p. 9325-36.

35. Hasan, N. A., et al., Microbial community profiling of human saliva using shotgun metagenomic sequencing. PLoS One, 2014. 9(5): p. e97699.

36. Ponnusamy, D., et al., Cross-talk among flesh-eating Aeromonas hydrophila strains in mixed infection leading to necrotizing fasciitis. Proc Natl Acad Sci USA, 2016. 113(3): p. 722-7.

37. Bankevich, A., et al., SPAdes: a new genome assembly algorithm and its applications to single-cell sequencing. Journal of computational biology, 2012. 19(5): p. 455-477.

38. Hyatt, D., et al., Prodigal: prokaryotic gene recognition and translation initiation site identification. BMC bioinformatics, 2010. 11(1): p. 119.

39. Zerbino, D. R. and E. Birney, Velvet: Algorithms for de novo short read assembly using de Bruijn. Genome Research, 2004.

40. Sumner, L. W., et al., Proposed minimum reporting standards for chemical analysis. Metabolomics, 2007. 3(3): p. 211-221.

41. Husby, S., et al., European society paediatric gastroenterology, hepatology and nutrition guidelines for diagnosing coeliac disease 2020. Journal of Pediatric Gastroenterology and Nutrition, 2020. 70(1): p. 141-156.

42. Yoshida, N., et al., Bacteroides vulgatus and Bacteroides dorei reduce gut microbial lipopolysaccharide production and inhibit atherosclerosis. Circulation, 2018. 138(22): p. 2486-2498.

43. Haghikia, A., et al., Dietary fatty acids directly impact central nervous system autoimmunity via the small intestine. Immunity, 2015. 43(4): p. 817-829.

44. Huang, Q., et al., Children Developing Celiac Disease Have a Distinct and Proinflammatory Gut Microbiota in the First 5 Years of Life. bioRxiv, 2020.

45. Stewart, C. J., et al., Temporal development of the gut microbiome in early childhood from the TEDDY study. Nature, 2018. 562(7728): p. 583.

46. Nakanishi, Y., T. Sato, and T. Ohteki, Commensal Gram-positive bacteria initiates colitis by inducing monocyte/macrophage mobilization. Mucosal immunology, 2015. 8(1): p. 152-160.

47. Sokol, H., et al., Low counts of Faecalibacterium prausnitzii in colitis microbiota. Inflammatory bowel diseases, 2009. 15(8): p. 1183-1189.

48. Machiels, K., et al., A decrease of the butyrate-producing species Roseburia hominis and Faecalibacterium prausnitzii defines dysbiosis in patients with ulcerative colitis. Gut, 2014. 63(8): p. 1275-1283.

49. Fite, A., et al., Longitudinal analyses of gut mucosal microbiotas in ulcerative colitis in relation to patient age and disease severity and duration. Journal of clinical microbiology, 2013. 51(3): p. 849-856.

50. Madsen, R. K., et al., Diagnostic properties of metabolic perturbations in rheumatoid arthritis. Arthritis research & therapy, 2011. 13(1): p. R19.

51. Monagas, M., et al., Dihydroxylated phenolic acids derived from microbial metabolism reduce lipopolysaccharide-stimulated cytokine secretion by human peripheral blood mononuclear cells. British journal of nutrition, 2009. 102(2): p. 201-206.

52. Wong, C. B., N. Iwabuchi, and J.-z. Xiao, Exploring the Science behind Bifidobacterium breve M-16V in Infant Health. Nutrients, 2019. 11(8): p. 1724.

53. Maerz, J. K., et al., Bacterial immunogenicity is critical for the induction of regulatory B cells in suppressing inflammatory immune responses. Frontiers in Immunology, 2020. 10: p. 3093.

54. Chung, W. S. F., et al., Prebiotic potential of pectin and pectic oligosaccharides to promote anti-inflammatory commensal bacteria in the human colon. FEMS microbiology ecology, 2017. 93(11): p. fix127.

55. Shimizu, J., et al., Propionate-producing bacteria in the intestine may associate with skewed responses of IL10-producing regulatory T cells in patients with relapsing polychondritis. PloS one, 2018. 13(9).

56. Leonard, M. M., et al., RNA sequencing of intestinal mucosa reveals novel pathways functionally linked to celiac disease pathogenesis. PloS one, 2019. 14(4).

57. Wei, Y., et al., Alterations of gut microbiome in autoimmune hepatitis. Gut, 2020. 69(3): p. 569-577.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising one, two, three, four, or more microbes listed in Table B, in a physiologically acceptable carrier and proper culture medium.

2. The composition of claim 1, comprising one, two, three, or more of:
Bacteroides ovatus CL03T12C18;
Clostridium sp JCC
a Faecalibacterium prausnitzii, preferably Faecalibacterium prausnitzii L2_6;
at least one Bifidobacterium longum strain, preferably selected from the group consisting of: Bifidobacterium longum DJO10A, Bifidobacterium longum NCC2705, Bifidobacterium longum subsp infantis CCUG 52486, Bifidobacterium longum subsp longum 1 6B, Bifidobacterium longum subsp longum 17 1B, Bifidobacterium longum subsp longum 35B, Bifidobacterium longum subsp longum 72B, Bifidobacterium longum subsp longum ATCC 55813, Bifidobacterium longum subsp longum F8, Bifidobacterium longum subsp longum GT15, and Bifidobacterium longum subsp longum KACC 91563; and
at least one Bifidobacterium breve strain, preferably selected from the group consisting of Bifidobacterium breve 31L; Bifidobacterium breve 689b; Bifidobacterium breve ACS_071_V_Sch8b; Bifidobacterium breve CECT_7263; Bifidobacterium breve DPC_6330; Bifidobacterium breve HPH0326; Bifidobacterium breve JCM 7017; Bifidobacterium breve JCM 7019; and Bifidobacterium breve S27.

3. A composition comprising one, two, three, four, or more anti-inflammatory metabolites selected from the group consisting of 2-Hydroxy-3-methylbutyric acid; Acetyl galactosamine; 2-Hydroxyisocaproic acid; Arabinonic acid; Lauric acid; 3-Hydroxyphenylacetic acid; Ribitol; Gluconic acid; Proline; Glycine; Glycerol; and Serine, in a physiologically acceptable carrier.

4. The composition of claim 3, comprising 2-Hydroxy-3-methylbutyric acid; Acetyl galactosamine; 2-Hydroxyisocaproic acid; and Arabinonic acid.

5. The composition of claim 1, which is formulated for oral administration.

6. The composition of claim 5, which is a liquid, capsule, gel, or tablet.

7. (canceled)

8. (canceled)

9. A method of treating or reducing the risk of developing a chronic inflammatory condition, the method comprising administering to a subject in need thereof an effective amount of the composition of claim 1.

10. The method of claim 9, wherein the chronic inflammatory condition is celiac disease.

11. A method of determining risk of developing a chronic inflammatory condition in a subject, the method comprising:
providing a sample comprising stool from the subject;
performing an assay to detect presence or level of a pro-inflammatory biomarker comprising
(i) at least one, two, three, four, or more pro-inflammatory microbes listed in Table A, and/or
(ii) at least one, two, or all three pro-inflammatory metabolites comprising serine, threonine, and glycolic acid;
comparing the presence or level of the pro-inflammatory biomarker in the sample to the presence or level of the pro-inflammatory biomarker in a reference sample; and
identifying a subject who has a presence or level of the pro-inflammatory biomarker above the reference sample as being at risk of developing the chronic inflammatory condition, e.g., within 6, 12, 18, or 24 months.

12. The method of claim 11, wherein the pro-inflammatory microbes comprise two, three, four, or more of Porphyromonas sp., preferably Porphyromonas_sp_31_2; an Alistipes finegoldii species, preferably Alistipes finegoldii_DSM_17242; a Ruminococcus bicirculans species; Alistipes_sp_HGB5; Erysipelotrichaceae_bacterium_21_3; a Dialister invisus species, preferably Dialister_invisus_DSM_15470; a Veillonella parvula, preferably Veillonella_parvula_ACS_068_V_Sch12 or Veillonella_parvula_HSIVP1; a Parabacteroides sp., preferably Parabacteroides_sp_20_3, or Parabacteroides_sp_D13; a Lachnospiraceae bacterium species, preferably Lachnospiraceae_bacterium_3_1_46FAA; and/or Bifidobacterium adolescentis species, preferably Bifidobacterium_adolescentis_L2_32.

13. The method of claim 11, further comprising treating the subject identified as being at risk to reduce the risk of developing the disease.

14. The method of claim 13, wherein treating the subject comprises administering an effective amount of the composition of claims 1 to 4.

15. The method of claim 14, wherein the composition is administered orally.

16. The method of claim 15, wherein the composition is a liquid, capsule, gel, or tablet.

17. The method of claim 11, wherein performing an assay to detect presence or level of a pro-inflammatory microbe comprises performing an assay to detect a protein or nucleic acid that identifies the microbe.
